(19) United States
(12) Patent Application Publication
(10) Pub. No.: US 2016/0278647 A1
(43) Pub. Date: Sep. 29, 2016

(54) MISALIGNMENT DETECTION OF A WEARABLE DEVICE

(71) Applicant: Intel Corporation, Santa Clara, CA (US)

(72) Inventors: Brian K. Vogel, Santa Clara, CA (US); John C. Wei, Saratoga, CA (US); Fai Angus Yeung, San Jose, CA (US)

(21) Appl. No.: 14/670,284

(22) Filed: Mar. 26, 2015

(51) Int. Cl. A61B 5/024; A61B 5/00

(52) U.S. Cl. CPC A61B 5/02438; A61B 5/6817; A61B 5/7221; A61B 5/6824; A61B 5/6823; A61B 5/7264; A61B 5/7405; A61B 2560/0266

(57) ABSTRACT

Technology for detecting whether a wearable device is misaligned is disclosed. The device can include a number of sensors, such as heart rate, temperature, or other sensors used to sense a physiologic aspect of the user, such as heart rate and can further contain components capable of providing data as to the proper alignment or placement of the wearable device on the user. The wearable device may communicate with a computing device, such as a mobile device which can receive data from the wearable device and output notifications to the user, including notifications about proper or improper placement or alignment of the wearable device.

### TECHNICAL FIELD

Technology described herein relates generally to wearable electronic devices.

### BACKGROUND

The popularity of wearable technology, such as smart watches and smart eyewear, has grown in recent years. Wearable technology can include clothing or accessories that incorporate computer and electronic technologies. Wearable technology can perform a variety of functions that are beneficial to a user, in addition to being aesthetically pleasing. For example, wearable technology can provide health monitoring and health metrics, music listening, global positioning system (GPS) capabilities, activity tracking, telephony services, internet browsing, etc. while the user is wearing the wearable technology.

### BRIEF DESCRIPTION OF THE DRAWINGS

Features and advantages of invention embodiments will be apparent from the detailed description which follows, taken in conjunction with the accompanying drawings; and, wherein:

FIG. 1 illustrates a heart rate monitoring headphone communicating heart rate information to a mobile device in accordance with an example;

FIG. 2 illustrates a system and related operations for detecting whether a wearable device is misaligned in accordance with an example;

FIGS. 3A and 3B illustrate acceleration vectors in a three dimensional (3D) coordinate system in accordance with an example;

FIG. 4 illustrates a flow chart for detecting whether a wearable device is misaligned in accordance with an example;

FIG. 5 depicts functionality of a mobile device operable to detect whether a wearable device is currently worn, and if so, to also detect its state of alignment in accordance with an example;

FIG. 6 depicts functionality of a wearable heart rate monitor in accordance with an example;

FIG. 7 depicts functionality of a system that includes a wearable heart rate monitoring device and a mobile device in accordance with an example;

FIG. 8 depicts a flow chart of a method for generating notifications for a heart rate monitoring device in accordance with an example; and

FIG. 9 illustrates a diagram of a computing device in accordance with an example.

Reference will now be made to the exemplary invention embodiments illustrated, and specific language will be used herein to describe the same. It will nevertheless be understood that no limitation of the scope of the disclosure is thereby intended.

### DETAILED DESCRIPTION

Before the various invention embodiments are disclosed and described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics may be combined in any suitable manner in one or more embodiments. In the following description, numerous specific details are provided, such as examples of layouts, distances, network examples, etc., to provide a thorough understanding of various invention embodiments. One skilled in the relevant art will recognize, however, that such detailed embodiments do not limit the overall inventive concepts articulated herein, but are merely representative thereof.

### Example Embodiments

An initial overview of technology embodiments is provided below and then specific technology embodiments are described in further detail later. This initial summary is intended to aid readers in understanding the technology more quickly, but is not intended to identify key or essential technological features nor is it intended to limit the scope of the claimed subject matter. Unless defined otherwise, all technical and scientific terms used herein have the same meaning as commonly understood by one of ordinary skill in the art to which this disclosure belongs.

Reference throughout this specification to “an example” means that a particular feature, structure, or characteristic described in connection with the example is included in at least one invention embodiment. Thus, appearances of the phrases “in an example” in various places throughout this specification are not necessarily all referring to the same embodiment.

As used in this specification and the appended claims, the singular forms “a,” “an” and “the” include plural referents unless the context clearly dictates otherwise. Thus, for example, reference to “a layer” includes a plurality of such layers.

In this specification, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising,” “including,” or “having” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for the exact number or numerical range without the term “about”. For example, for the sake of convenience and brevity, a numerical range of “about 50 angstroms to about 80 angstroms” should also be understood to provide support for the range of “50 angstroms to 80 angstroms.”

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and invention examples may be referred to herein along with alternatives for the various components thereof. It is understood that such embodiments, examples, and alternatives are not to be construed as defacto equivalents of one another, but are to be considered as separate and autonomous representations of various invention embodiments.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, in reciting the range of “about 1 to about 5,” support is given for all sub-ranges within the range such as from 1-3, from 2-4, from 3.5-4.5 and from 3-5, etc., as well as the individual numbers 1, 2, 3, 4, and 5 and further including portions or fractions thereof such as 2.5, 3.6, 4.8, 1¾, 3¼, and 4½.

This disclosure describes a technology for determining whether a wearable sensor device (e.g., a heart rate monitoring device) is currently worn, and if so, whether the wearable device is aligned or misaligned with a body feature or surface of a user. Stated differently, the technology is for determining whether a wearable sensor device is worn by a user and is placed or positioned correctly so as to receive and collect accurate data from the user.

In one example, the wearable device can be inserted in a user's ear and used to monitor the user's heart rate. Some sensors in the wearable device are “physiologic sensors” and are used to detect a targeted physiologic action, condition, parameter, or property, such as heart rate, perspiration rate, respiration rate, or temperature. Other sensors in the device are “alignment sensors” used to detect the alignment or orientation of the wearable sensor device when engaged with a user. Exemplary “alignment sensors” include without limitation, accelerometers, gyroscopes, proximity sensors, pressure sensors, etc. In some embodiments, a “physiologic sensor” can be used to aid alignment and therefore also classified as an “alignment sensor”. For example, a temperature sensor can aid in determining proper alignment of a wearable heart rate monitor configured to be placed in an individual's ear to monitor heart rate. If the temperature sensed is within a normal range for the individual, then such parameter would support a conclusion of proper alignment. However, if the temperature is outside of a normal range for the individual, then such parameter would support misalignment.

The sensor data can be communicated from the wearable sensor device to a computing device (e.g., a mobile computing device) via a communication link between the computing device and the wearable sensor device. Such link can be either a wired or wireless connection. Once collected, the sensor data can be provided to a classifier. The classifier (e.g., a linear classifier) can compare the sensor data to historical data to determine whether the wearable sensor device is currently worn. Collected information that does not correspond with expected values supports a determination of misalignment and collected information that does correspond with expected values supports a determination of proper alignment. When misalignment is determined, a notification can be generated. In one embodiment, the notification can be audible. In another embodiment, the notification can be visual, for example a visual notification on a display of the computing device.

In some cases, misalignment may be due to improper sizing or fit of the wearable sensor device. In such cases the misalignment notification can include recommendations for wearable sensor devices of an alternative size. By varying the size of the wearable sensor device to enable an improved fit within the individual's ear, an accuracy of the physiologic sensor values can improve.

One example of a wearable sensor device is smart earbuds (or smart headphones) with heart rate sensing or monitoring functionality. Once detected, the heart rate information can be provided to the user. An exemplary mechanism for providing the information is via a mobile application on a mobile computing device, such as a smart phone that is in communication with the smart earbuds. In some examples, the smart earbuds can be available in various sizes, such as small, medium or large. Medium size earbuds may be a good fit for a majority of users and therefore provide accurate heart rate information. However, for some users medium size earbuds fit poorly and thus provide inaccurate heart rate information. For these users, small or large size earbuds may be a better fit and thus provide more accurate heart rate information. Unfortunately, users often are unaware that the improper fit of the earbuds is the reason for detection inaccuracies and erroneously deem the product defective. This typically results in returning the product to the merchant from which it was purchased. However, if the user were aware that the reason for detection inaccuracies was merely due to improper sizing, then a correct size could be selected and the return of the product to the merchant could be avoided.

There can be a number of other reasons for earbud misalignment, such as the user's movement or activity, improper insertion into the ear, tension on an earbud cord, perspiration that causes slippage or a combination of like factors. Misalignment prevents heart rate sensors in the earbuds from establishing a proper relationship, such as a contact relationship, with key areas inside the user's ear. Therefore, inaccurate heart rate information can result. On the other hand, when the earbuds are aligned properly within the ear, heart rate sensors can achieve a proper relationship with the key areas and effectively monitor pulses of blood through the blood vessels inside the ear.

Detection of misfit and/or misalignment of a wearable sensor device can be performed by collecting alignment sensor data, such as accelerometer data, proximity data, pressure data, or temperature data. The alignment sensor data can be used to detect whether the wearable device is currently in use (e.g. in the ear) and whether it is misaligned from a position required for accurate data collection. In addition, collected physiologic sensor data, such as heart rate information, can be compared to historical data to determine whether the collected data is within an acceptable range. Other modeling parameters such as typical motion range and a natural orientation of the wearable sensor device can further aid in determining whether the device is properly worn and aligned. A notification can be generated so users can correct fit and alignment problems with the wearable sensor device. In one aspect, the notification can be generated for display at a mobile computing device with which the wearable sensor device communicates. In another aspect, the notification can be a visual or audible signal made by the wearable sensor device itself.

In one example, information from alignment sensors in heart rate sensing earbuds can be used to determine whether the earbuds are located within a user's ear and/or properly aligned. Since the user's head is physically constrained to moving with a range of 180 degrees horizontally and 90 degrees vertically, acceleration values from accelerometers that characterize movement must be relatively minimal and have a short duration. Additionally, proximity values from proximity sensors will indicate relatively close contact between the earbuds and the user's ear. Furthermore, pressure and temperature values collected by pressure and temperature sensors will be within an acceptable range. The standard values against which the detected information is compared can be pre-established based on general population data, can be established through a user's prior use, or both.

When the wearable sensor or detector device takes the form of earbuds, it can have components required to broadcast or transmit music or other audio signals to the ear of a user. For example, an electrically conductive line or cable (i.e. cord) having a speaker at one end and an audio jack at the other end can be included. In such an embodiment, the audio jack will be configured to plug into a corresponding receiver on a mobile computing device and receive electronic signal therefrom. Other components required to function as typical earbuds can also be included. In some embodiments, the earbuds may receive power to operate through the cable connecting the earbuds to the mobile computing device. In other embodiments the earbuds, or other wearable sensor device, can include its own power source. Earbuds can detect a user's heart rate either with or without music playing through the earbuds. Additional components or features for earbud performance or the comfort of a user can also be included such as padded tips or gel moldings which help improve a friction fit within the user's ear.

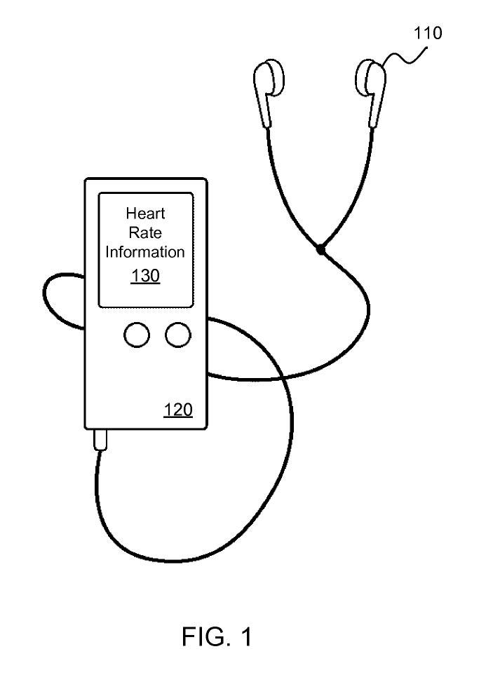

FIG. 1 illustrates an exemplary wearable sensor device and system for detecting and reporting physiologic information of an individual. In this case, the wearable sensor device takes the form of a heart rate sensing or monitoring headphone or earbud, 110 which can communicate heart rate information 130 to a mobile computing device 120. The heart rate monitoring headphone 110 can also be referred to as smart earbuds or earbuds that are capable of detecting heart rate information. The heart rate monitoring headphone 110 can include a heart rate sensor (not shown in FIG. 1) that collects heart rate information from a user's ear 130 and sends the heart rate information to the mobile computing device 120. In one example, the heart rate information 130 can be sent via a wired connection between the heart rate monitoring headphone 110 and the mobile computing device 120. In an alternative example, heart rate monitoring headphone 110 can be a wireless headphone and the heart rate information 130 can be wirelessly communicated from the heart rate monitoring headphone 110 to the mobile computing device 120. In some embodiments, wireless communication of heart rate information can occur even when the heart rate monitoring headphone includes a cord and is plugged into the mobile computing device 120. The mobile computing device 120 can display the heart rate information 130 of the individual. In an additional embodiment (not shown) the heart rate information can be communicated audibly through the headphone speaker. In yet other embodiments, the wearable sensor device can be a heart rate monitor worn on the chest or other portion of the body and be either wired or wireless.

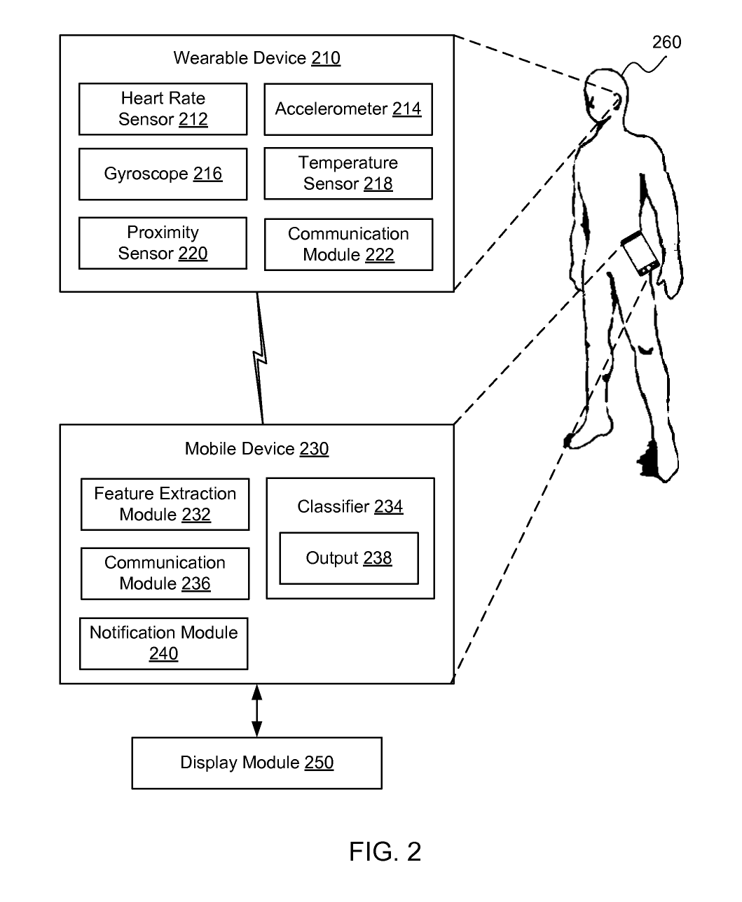

FIG. 2 illustrates an exemplary system for detecting a user's heart rate using a wearable sensor device 210. The wearable device 210 can include an earbud speaker that is capable of providing audio to a user 260. The wearable device 210 can include a heart rate sensor 212 with heart rate monitoring capability. The heart rate sensor 212 can detect the heart rate of the user 260 whether or not audio is being provided. By integrating the heart rate sensor 212 with the wearable computing device 210, the user 260 does not need to rely on additional devices for monitoring heart rate. However, in some embodiments, the in-ear heart rate sensor may be used in combination with an additional heart rate sensor, for example a heart rate sensor which is worn on the user's chest or wrist. In such embodiment, data from each sensor can be used to confirm the accuracy of the other sensor, or combined to improve overall detection and reporting accuracy.

The technology described herein additionally provides methods for determining when the wearable sensor device 210 is misaligned with a body feature or surface of the user 260. The wearable sensor device 210 can be determined as being misaligned based on sensor data acquired by alignment sensors (not shown) in the wearable sensor device 210. If the wearable sensor device 210 is misaligned, then the heart rate sensor 212 can collect inaccurate heart rate measurements from the user 260. A notification can be generated for display on the mobile computing device 230 when the wearable sensor device 210 is improperly positioned or otherwise misaligned. In some aspects, the notification can instruct the user 260 to adjust a position of the wearable sensor device 210. In addition, the notification can include recommendations for alternative sizes of the wearable sensor device 210 (e.g., small, medium, large) in order for the user 260 to obtain more accurate heart rate information. In one embodiment, the notification can be an audible message broadcast to the user 260 through the headphone speakers. Such message may simply be a tone or alarm, or can include verbal instructions regarding the misalignment and in some embodiments, of how to adjust the device in order to achieve correct alignment or location and/or recommended size adjustment. Furthermore, an indication of proper alignment can be given, either audibly or visually upon adjusting the wearable sensor device 210 into proper alignment.

Various types of alignment sensor data can be used in order to determine whether the wearable sensor device 210 is misaligned with the user's body feature or surface. As with the heart rate or other physiologic sensor data, the alignment sensor data can also be communicated from the wearable sensor device 210 to the mobile computing device 230 via a wired or a wireless connection. The mobile computing device 230 can determine whether the wearable sensor device 210 is currently worn and/or misaligned based on the alignment sensor data. Indication of misalignment or improper placement can be made using the mechanisms previously mentioned, such a visual or audible notification either from the mobile computing device 230 or the wearable sensor device 210.

As mentioned, one type of alignment sensor in the wearable computing device 210 can be an accelerometer 214. The accelerometer can collect acceleration data for the wearable sensor device 210 by detecting inertial forces in the X, Y and Z components. The acceleration data can include a plurality of acceleration vectors that the accelerometer 214 measures at particular time instances. For example, the acceleration data can include a first acceleration vector (R1) at a first time instance and a second acceleration vector (R2) at a second time instance. The first acceleration vector and the second acceleration vector can be sampled in succession, for example, according to a sample rate of the accelerometer 214. In other words, the accelerometer 214 can consecutively measure the first acceleration vector and the second acceleration vector at consecutive time instances.

The wearable sensor device 210 can send the acceleration data to the mobile device 230 via a communication module 222 that is included in the wearable sensor device 210. Similarly, the mobile computing device 230 can receive the acceleration data via a communication module 236 installed on the mobile computing device. The acceleration data received by the mobile device 230 can include an amount of acceleration of the wearable device 210 at each instance between samples collected from the accelerometer 214.

The wearable device 210, via a feature extraction module 232, can calculate a change in magnitude (d) between the first acceleration vector (R1) and the second acceleration vector (R2). The change in magnitude (d) can be a first feature extracted from the acceleration data. The change in magnitude (d) can be estimated at the sample rate of the accelerometer 214. The change in magnitude (d) can indicate how much the wearable device 210 has moved over a period of time (irrespective of the wearable device's orientation).

The feature extraction module 232 can calculate the change in magnitude (d) using |\sqrt{R₂ₓ²+R_{2y}²+R_{2z}²}−\sqrt{R₁ₓ²+R_{1y}²+R_{1z}²}|, wherein R₁ₓ, R_{1y}, and R_{1z} are measured values associated with the first acceleration vector (R₁) for the X, Y and Z components, and R₂ₓ, R_{2y}, and R_{2z} are measured values associated with the second acceleration vector (R₂) for the X, Y and Z components. In one example, the acceleration data used to calculate the change in magnitude (d) can be previously passed through a high pass filter in order to remove a gravity component. Therefore, the change in magnitude (d) can correspond to the magnitude of a high-pass filtered acceleration magnitude, and does not consider an orientation component.

The change in magnitude (d) between the two acceleration vectors can generally be moderate when the wearable sensor device 210 is worn under typical conditions. In other words, the change in magnitude (d) can generally be within a defined range when the user 260 is using the wearable device 210 in a conventional fashion. On the other hand, the change in magnitude (d) can be substantially zero when the wearable sensor device 210 is currently resting on a flat surface (i.e., the wearable device 210 is not moving), and the change in magnitude (d) may not be moderate when the user 260 is taking off or putting on the wearable device 210. For example, the change in magnitude (d) can be outside the defined range when the user 260 is taking off or putting on the wearable device 210.

The feature extraction module 232 can provide the change in magnitude (d) to a classifier 234. In one example, the classifier 234 includes a linear classifier. The classifier 234 can compare the change in magnitude (d) to historical data. The historical data can include a plurality of changes in magnitude that correspond to instances when the wearable device 210 was worn or not worn. The historical data can be used to train the classifier 234. Based on the comparison, the classifier 234 can determine whether the wearable sensor device 210 is likely to be currently worn or not. The classifier 234 can determine that the wearable sensor device 210 is not currently worn when the change in magnitude (d) indicates that the wearable sensor device 210 is resting on a flat surface based on the comparison with the historical data. Similarly, the classifier 234 can determine that the wearable sensor device 210 is not currently worn when the change in magnitude (d) is outside typical changes in magnitude from historical data (e.g., which can imply that the user 260 is currently taking off or putting on the wearable sensor device 210). On the other hand, when the change in magnitude (d) for the wearable sensor device 210 is in line with typical changes in magnitude that occur when wearable sensor devices are appropriately worn, then the classifier 234 can infer that the wearable sensor device 210 might be currently worn by the user 260. As described in further detail below, the classifier 234 can also utilize other information when determining whether the wearable sensor device 210 is currently worn or not worn by the user 260. Therefore, the change in magnitude (d) can be one of several factors used to determine whether the wearable sensor device 210 is currently worn.

In one example, the mobile computing device 230 can receive acceleration data from the wearable sensor device 210 as part of a process for determining whether the wearable sensor device is worn by a user or not. The wearable sensor device 210, via the feature extraction module 232, can provide the acceleration data to a low pass filter in order to obtain low-pass filtered acceleration vectors. The low pass filtering can remove motion from an acceleration vector, thereby resulting in a gravity component from the acceleration data. The gravity component of the acceleration data can be a second feature extracted from the acceleration data. The gravity component can generally refer to the vector pointing to the center of the Earth. In addition, the gravity component of the acceleration data can provide a relative orientation of the wearable sensor device 210.

The feature extraction module 232 can provide the gravity component of the acceleration data to the classifier 234. The classifier 234 can compare the gravity component of the acceleration data to historical data. The historical data can include a plurality of relative orientations that correspond to instances when wearable sensor devices were worn or not worn. Based on the comparison, the classifier 234 can determine whether the wearable sensor device 210 is likely to be currently worn or not.

In one example, the classifier 234 can be trained using known possible orientations and/or angles of the wearable sensor device 210. Physical constraints can limit the rotation of the user's head with respect to the gravity component. The approximate gravity angles can be known when the user 260 is using the wearable sensor device 210 in a typical manner (e.g., standing up, sitting down, lying down, or running). In other words, the classifier 234 can be trained using an acceptable range of known reference angles. The reference angle does not generally exceed more than approximately 90 degrees from the reference. For example, in order for the wearable sensor device's orientation or angular position to change by 180 degrees, the user's head would be upside down (e.g., which could occur when the user 260 is standing on their head), but these situations are unlikely to be typical usage conditions.

Therefore, when the user 260 is sitting, standing, etc. when using the wearable sensor device 210, the gravity component of the acceleration data is generally within an acceptable range. Even if the user 260 is relatively still, there are generally some minimal changes in the gravity component over a period of time (e.g., several seconds). In these situations, the classifier 234 can determine that the wearable sensor device 210 is currently worn by the user 260. On the other hand, if the gravity component indicates substantially no change over the period of time, then the classifier 234 can infer that the wearable sensor device 210 is not currently worn by the user 260. In addition, if the gravity component indicates relative orientations of the wearable device 210 that are implausible if worn, the classifier 234 can infer that the wearable sensor device 210 is not currently worn by the user 260.

In one example, the mobile computing device 230 can receive acceleration data from the wearable sensor device 210 as part of a process of determining proper alignment or misalignment of the device when worn by a user. As previously described, the acceleration data can include the first acceleration vector (R1) and the second acceleration vector (R2). The acceleration data can be passed through a high pass filter in order to remove a gravity component from the acceleration data, which results in acceleration magnitude data. If the wearable sensor device 210 is misaligned in the user's ear, then a change of coordinate system is caused and the first acceleration vector (R1) becomes the second acceleration vector (R2). If the wearable sensor device 210 is not at a resting position, then a magnitude of R2 can deviate from a magnitude of R1. In other words, the wearable sensor device 210 may not be resting properly within the user's ear. The angle between R1 and R2 can be defined as ø, which can denote an angular deviation of a new force vector.

The wearable sensor device 210, via the feature extraction module 232, can calculate a misalignment vector (r) as a difference between the first acceleration vector (R1) and the second acceleration vector (R2). The misalignment vector can be a third feature extracted from the acceleration data. The feature extraction module can calculate the misalignment vector (r) using (R₁ₓ−R₂ₓ, R_{1y}−R_{2y}, R_{1z}−R_{2z}), wherein R₁ₓ, R_{1y}, and R_{1z} are measured values associated with the first acceleration vector (R₁); and R₂ₓ, R_{2y}, and R_{2z} are measured values associated with the second acceleration vector (R₂). In one example, the misalignment vector (r) can be more responsive to misfit of the wearable sensor device 210 as compared to the angular deviation (0) because the misalignment vector (r) takes into account both angular and magnitude deviations using the acceleration data.

The feature extraction module 232 can provide the misalignment vector to the classifier 234. The classifier 234 can compare the misalignment vector to historical data. The historical data can include a plurality of angular and magnitude deviations over a period of time. The angular and magnitude deviations in the historical data can correspond to wearable devices that were aligned or misaligned when currently worn. Based on the comparison, the classifier 234 can determine whether the wearable sensor device 210 is misaligned with a body feature or surface of the user 260. Therefore, the misalignment vector (r) can be used by the classifier 234 to determine whether the wearable sensor device 210 is misaligned and/or is an improper fit for the user 260.

In one example, the feature extraction module 232 can average the change in magnitude (d) or the misalignment vector (r) over a period of time. The feature extraction module 232 can provide the average to the classifier 234. The classifier 234 can compare the average of the change in magnitude (d) or the misalignment vector (r) to a defined threshold, wherein the defined threshold is a tunable parameter. Based on the comparison, the classifier 234 can determine whether the wearable sensor device 210 is currently worn and/or misaligned with the body feature or surface based on the average in relation to the defined threshold.

In one example, the wearable sensor device 210 can include a gyroscope 216. The gyroscope 216 can collect orientation measurements for the wearable sensory device 210 over a period of time. The wearable sensor device 210 can send the orientation measurements to the mobile computing device 230 via the communication module 222. The orientation measurements can be provided to the classifier 234 on the mobile device 230. The classifier 234 can compare the orientation measurements to historical data, and based on the comparison, the classifier 234 can infer whether the wearable device 210 is currently worn and/or misaligned with the user's body feature or surface.

In one example, the wearable sensor device 210 can include a temperature sensor 218. The temperature sensor 218 can collect temperature measurements for the wearable sensor device 210 over a period of time. In other words, the temperature sensor 218 can sense temperatures within the user's ear or other body surface. The wearable sensor device 210 can send the temperature measurements to the mobile computing device 230 via the communication module 222. The temperature measurements can be provided to the classifier 234 on the mobile computing device 230. The classifier 234 can compare the temperature measurements to historical data or a range of typical temperatures if the wearable device 210 is worn, and based on the comparison, the classifier 234 can infer whether the wearable device 210 is currently worn by the user 260. For example, if the temperature measurements are within the range of acceptable temperatures, then the classifier 234 can infer that the wearable device 210 is currently worn by the user 260.

In one example, the wearable sensor device 210 can include a proximity sensor 220. The proximity sensor 220 can collect proximity measurements for the wearable sensor device 210 over a period of time. In other words, a distance provided by the proximity sensor 218 can be approximately a distance between the proximity sensor 218 and a contact point of a sensor window. The wearable sensor device 210 can send the proximity measurements to the mobile computing device 230 via the communication module 222. The proximity measurements can be provided to the classifier 234 on the mobile computing device 230. The classifier 234 can compare the proximity measurements to historical data, and based on the comparison, the classifier 234 can infer whether the wearable sensor device 210 is currently worn by the user 260. In one example, the proximity sensor 220 can falsely detect that the wearable device 210 is currently worn when the wearable device 210 is outside the ear and sitting on a still surface (e.g., a table). In this situation, the temperature measurements can be an additional metric used to determine that the wearable device 210 is currently worn.

In one example, the wearable sensor device 210 can include a pressure sensor (not shown). The pressure sensor can collect pressure measurements for the wearable sensor device 210 over a period of time. In other words, a pressure on or around the wearable sensor device can be determined as a measure of force exerted on the device by the tissue within which it is in contact. The wearable sensor device 210 can send the pressure measurements to the mobile computing device 230 via the communication module 222. The pressure measurements can be provided to the classifier 234 on the mobile computing device 230. The classifier 234 can compare the pressure measurements to historical data, and based on the comparison, the classifier 234 can infer whether the wearable sensor device 210 is currently worn by the user 260. In one example, pressure measurements can be used an additional metric of determination when data from the other alignment sensors is unclear, such as proximity data from a proximity sensor when the wearable sensor device 210 is resting on a surface.

In one configuration, heart rate information collected by the heart rate sensor 212 can be provided to the classifier 234 on the mobile device 230. The classifier 234 can compare computed heart rates to an expected heart rate range. A confidence level can be used to quantify the computed heart rates in relation to the expected heart rate range. The confidence level can be a value derived from a frequency spectrum of sensed pulses from the proximity sensor 220. In one example, a bounding box can be centered at a target heart rate, such as 72 beats per minute (bpm). The confidence value can be estimated from the bounding box by counting the number of data points in the bounding box. If the user 260 is increasing the amount of cardiac activities, then the target heart rate and hence the center of the bounding box can move upward. If the computed heart rates are not within the expected heart rate range, then the classifier 234 can determine that the wearable sensor device 210 is misaligned with the user's body feature or surface. In other words, the misalignment between the wearable sensor device 210 and the user's ear can cause heart rate information to be collected for the user 260 that is physically implausible.

The wearable sensor device 210 can extract various features from the acceleration data in order to determine whether the wearable sensor device 210 is currently worn and/or misaligned with the user's body feature or surface. In one embodiment at least three features can be extracted from the acceleration data to determine whether the wearable sensor device 210 is currently worn and/or misaligned. For example, the first feature can include a high-pass filtered change in magnitude between two acceleration vectors, the second feature can include a high-pass filtered misalignment vector between the two acceleration vectors, and the third feature can include a low-pass filtered gravity component of the acceleration data. In addition, gyroscope measurements, temperature measurements, proximity measurements, pressure measurement and a confidence level of heart rate measurements can be other features extracted from sensor data collected at the wearable sensor device 210. Any number of other features not presently discussed, such as light sensors, or chemical sensors that can positively contribute to an accurate determination of whether a wearable sensor device 210 is worn by a user and/or properly aligned can be used.

The classifier 234 included in the wearable sensor device 210 can use a combination of the above features to determine whether the wearable sensor device 210 is currently worn and/or misaligned with the user's body feature or surface. For example, the classifier 234 can use a combination of the high-pass filtered change in magnitude between two acceleration vectors, the low-pass filtered gravity component of the acceleration data, temperature data, pressure data and proximity data in order to determine whether the wearable device 210 is currently worn by the user 260. In another example, the classifier 234 can use a combination of the high-pass filtered misalignment vector between the two acceleration vectors, gyroscope data and heart rate confidence levels to determine whether the wearable device 210 is currently worn and/or misaligned with the user's body feature or surface. The classifier 234 can be previously trained using historical data. The classifier 234 can compare the various features to the historical data when determining whether the wearable device 210 is currently worn and/or misaligned. Any combination of data types which aids in the determination of whether a wearable sensor device 210 is worn and/or aligned or misaligned can be used.

In one example, the classifier 234 can provide various outputs 238 depending on whether or not the wearable sensor device 210 is currently worn and aligned or misaligned. For example, the classifier 234 can provide a first output 238 indicating that the wearable device 210 is misaligned with the body feature or surface. The classifier 234 can provide a second output 238 indicating that the wearable sensor device 210 is not misaligned with the body feature or surface.

A notification module 240 can use the output 238 to generate a notification for display via the display module 250. For example, if the output 238 indicates that the wearable device 210 is misaligned with the user's body feature or surface, the notification module 240 can generate a notification indicating that a misalignment can be causing inaccurate heart rate information to be displayed to the user 260. In addition, the notification can include recommendations for improving the accuracy of the heart rate information. In one example, the notification can include instructions on how the user 260 can adjust the wearable sensor device 210 within the user's ear in order to obtain more accurate heart rate information. In another example, the notification can include suggestions to switch to an alternative size of the wearable device 210 (e.g., small, medium or large) in order to improve the accuracy of the heart rate information.

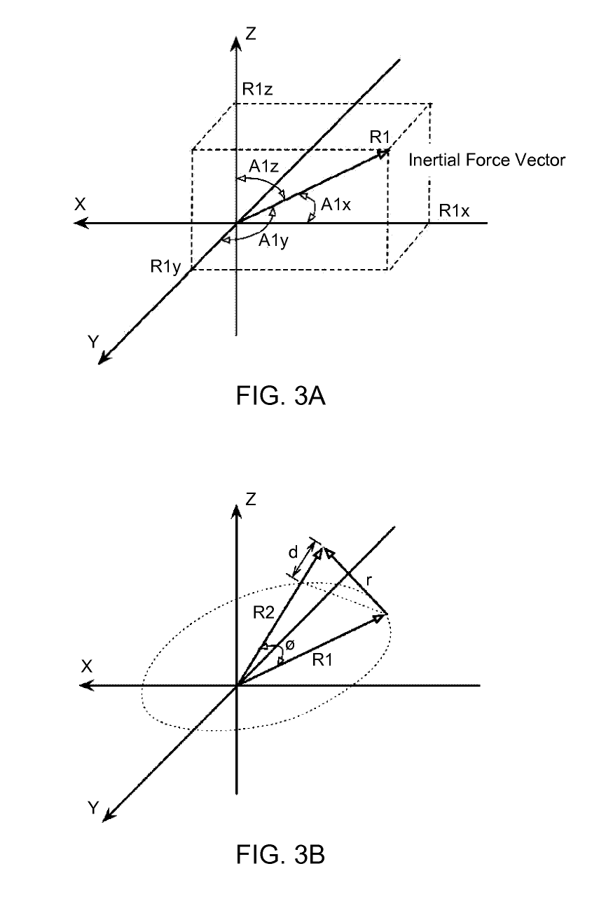

FIG. 3A illustrates an exemplary acceleration vector in a three dimensional (3D) coordinate system. The acceleration vector can be associated with a wearable device such as a wearable sensor device. As shown in FIG. 3A, a vector (R1) can be an acceleration vector that an accelerometer is measuring at a particular time instance. The acceleration vector (R1) can be represented by R₁ₓ, R_{1y} and R_{1z}. The acceleration vector (R1) can be related to R₁ₓ, R_{1y} and R_{1z}. based on the following relationship: R₁²=R₁ₓ²+R_{1y}²+R_{1z}². When the wearable device is at rest, then the resultant force vector (R1) is 1g. In other words, the inertial force is equivalent to the gravitational force. In addition, the angle between the acceleration vector (R1) and the X axis is represented by A1x, the angle between the acceleration vector (R1) and the Y axis is represented by A1y, and the angle between the acceleration vector (R1) and the Z axis is represented by A1z.

FIG. 3B illustrates exemplary acceleration vectors in a three dimensional (3D) coordinate system. The acceleration vectors can be associated with a wearable device. A first vector (R1) can be an acceleration vector that an accelerometer is measuring at a first time instance. A second vector (R2) can be an acceleration vector that an accelerometer is measuring at a second time instance. In other words, R2 can be measured after R1. In addition, R1 and R2 can be consecutive measurements of the accelerometer. An angle between R1 and R2 can be represented as ø. A misalignment vector (r) can represent a difference between R1 and R2. The misalignment vector (r) can be calculated using (R₁ₓ−R₂ₓ, R_{1y}−R_{2y}, R_{1z}−R_{2z}), wherein R₁ₓ, R_{1y}, and R_{1z} are measured values associated with R₁; and R₂ₓ, R_{2y}, and R_{2z} are measured values associated with the R₂. In addition, a change in magnitude (d) can be calculated between R1 and R2. The change in magnitude (d) can be calculated using |\sqrt{R₂ₓ²+R_{2y}²+R_{2z}²}−\sqrt{R₁ₓ²+R_{1y}²+R_{1z}²}|, wherein R₁ₓ, R_{1y}, and R_{1z} are measured values associated with R1 for the X, Y and Z components, and R₂ₓ, R_{2y}, and R_{2z} are measured values associated with R2 for the X, Y and Z components.

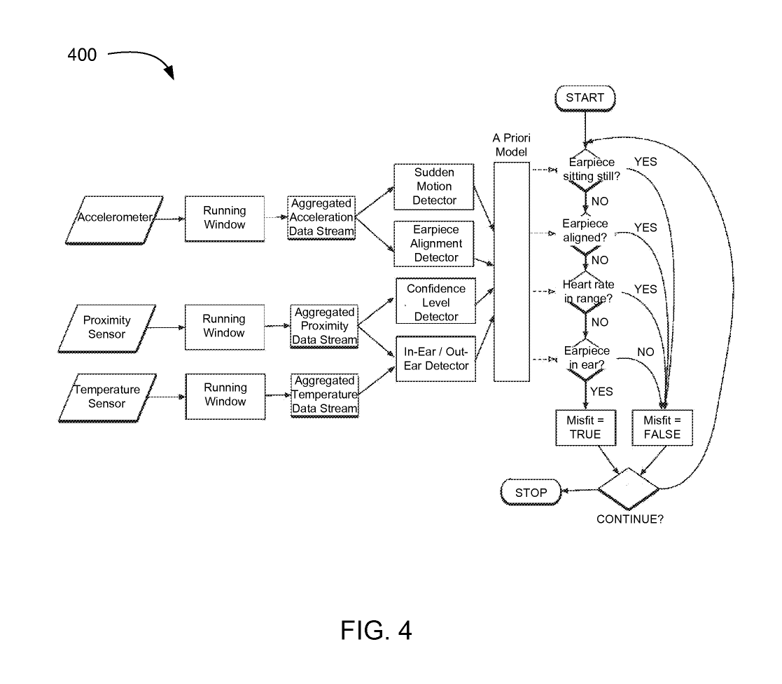

FIG. 4 illustrates an exemplary flow chart for detecting whether a wearable device, including a wearable sensor device is misaligned. The wearable device can include an accelerometer, a proximity sensor, a pressure sensor and a temperature sensor, for example. Data collected from the accelerometer can be aggregated over a running window (e.g., ten seconds) in order to create an aggregated acceleration data stream. Data collected from the proximity sensor can be aggregated over the running window (e.g., ten seconds) in order to create an aggregated proximity data stream. Data collected from the temperature sensor can be aggregated over the running window (e.g., ten seconds) in order to create an aggregated temperature data stream.

The aggregated acceleration data stream can be provided to a sudden motion detector. The sudden motion detector can compare the aggregated acceleration data stream to an a priori model in order to determine whether the wearable device is not moving. The aggregated acceleration data stream can be provided to an earpiece alignment detector. The earpiece alignment detector can compare the aggregated acceleration data stream to the a priori model in order to determine whether the wearable device is correctly aligned. The aggregated proximity data stream can be provided to a confidence level detector. The confidence level detector can compare the aggregated proximity data stream to the a priori model in order to determine whether heart rate information is within an acceptable range. The aggregated proximity data stream and the aggregated temperature data can be provided to an in-ear detector. The in-ear detector can compare the aggregated proximity data stream and the aggregated temperature data stream to the a priori model in order to determine whether the wearable device is inside the user's ear.

If the wearable device is moving, the wearable device is not correctly aligned, the heart rate information is not within the acceptable range, and the wearable device is inside the user's ear, then the wearable device can be detected as being a misfit. If the wearable device is not moving, the wearable device is correctly aligned, the heart rate information is within the acceptable range, and the wearable device is not inside the user's ear, then the wearable device can be detected as not being a misfit.

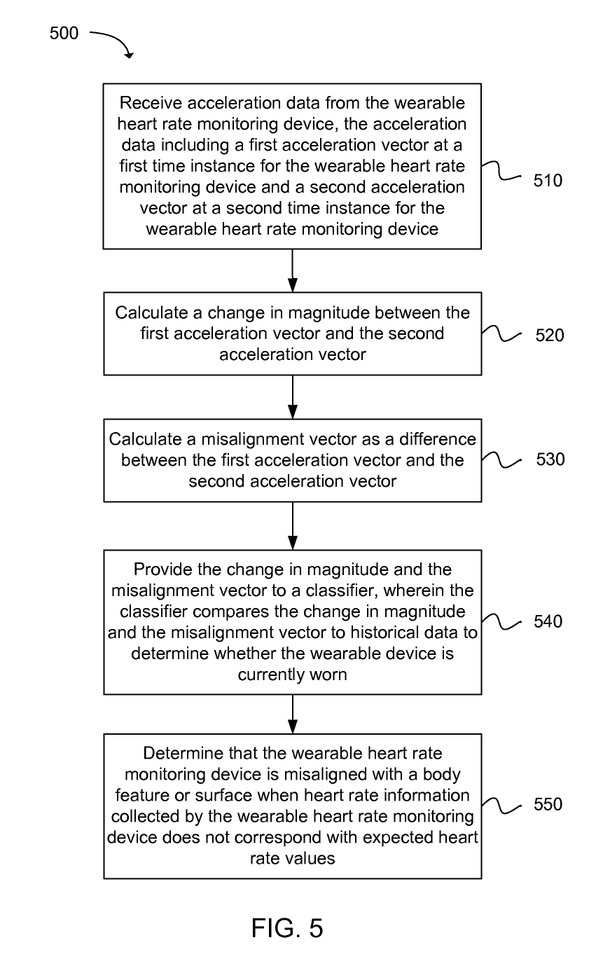

Another example provides functionality 500 of a mobile device comprising one or more processors configured to detect whether a wearable sensor device, such as a heart rate monitoring device is misaligned, as shown in the flow chart in FIG. 5. The functionality can be implemented as a method or the functionality can be executed as instructions on a machine, where the instructions are included on at least one computer readable medium or one non-transitory machine readable storage medium. The one or more processors can be configured to receive acceleration data from the wearable sensor device, the acceleration data including a first acceleration vector at a first time instance for the wearable heart rate monitoring device and a second acceleration vector at a second time instance for the wearable heart rate monitoring device, as in block 510. The one or more processors can be configured to calculate a change in magnitude between the first acceleration vector and the second acceleration vector, as in block 520. The one or more processors can be configured to calculate a misalignment vector as a difference between the first acceleration vector and the second acceleration vector, as in block 530. The one or more processors can be configured to provide the change in magnitude and the misalignment vector to a classifier, wherein the classifier compares the change in magnitude and the misalignment vector to historical data to determine whether the wearable sensor device is currently worn, as in block 540. The one or more processors can be configured to determine that the wearable sensor device is misaligned with a body feature or surface when heart rate information collected by the wearable sensor device does not correspond with expected heart rate values.

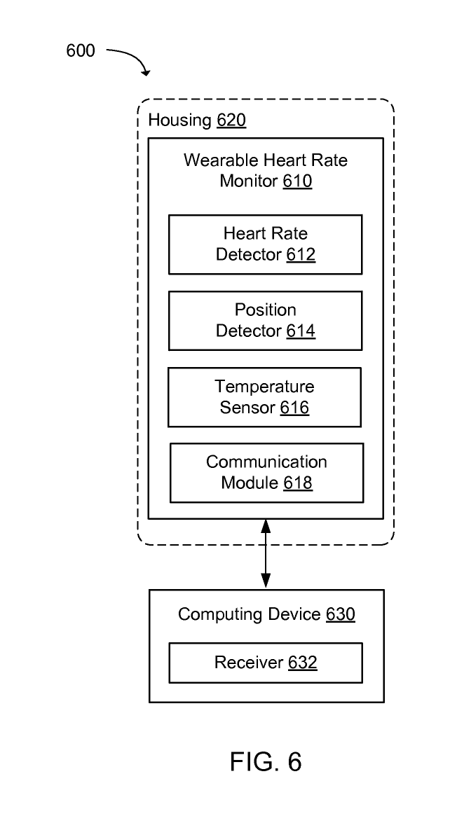

Another example provides functionality 600 of a sensor device, such as a wearable heart rate monitor, as shown in the block diagram in FIG. 6. The wearable sensor device 610 can include a heart rate detector 612, a position detector 614, and a temperature sensor 616. The wearable sensor device 610 can include a housing 620 configured to engage a body feature or surface in a manner that allows for heart rate detection. In addition, the wearable sensor device 610 can include a communication module 618 configured to transmit heart rate and position data to a receiver 632. The receiver 632 can be included in a computing device 630. As previously noted, in some embodiments where a mobile device is not used with the wearable sensor device, the additional processing components referred to as part of the mobile device can be included in the wearable sensor device itself.

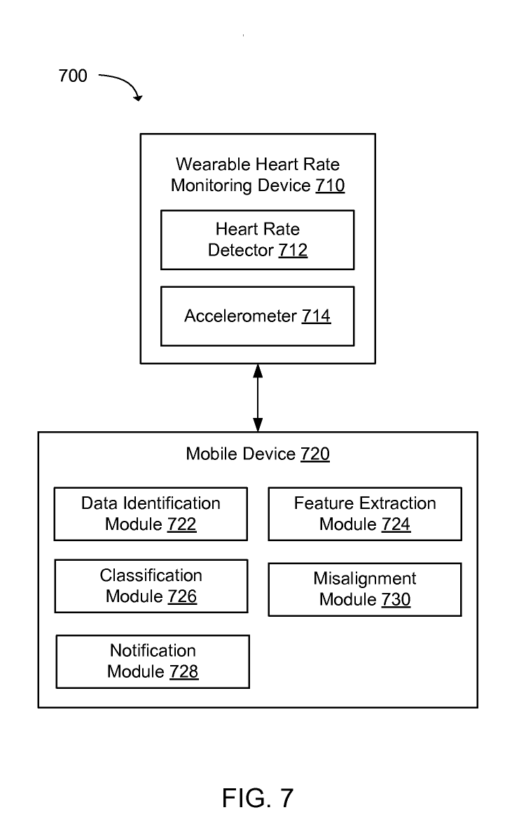

Another example provides a system 700 that includes a wearable sensor device, such as a heart rate monitoring device 710 and a mobile computing device 720, as shown in the block diagram in FIG. 7. The wearable sensor device can have a heart rate detector 712 and an accelerometer 714. The mobile computing device 720 can be in communication with the wearable device 710. The mobile computing device 720 can include a data identification module 722 configured to receive acceleration data for the wearable sensor device. The mobile computing device 720 can include a feature extraction module 724 configured to determine one or more features of the acceleration data. The mobile computing device 720 can include a classification module 726 configured to compare the one or more features of the acceleration data to historical data to determine whether the wearable sensor device is currently worn. The mobile computing device 720 can include a misalignment module 728 configured to determine that the wearable sensor device is misaligned with a body feature or surface when heart rate information collected by the heart rate detector does not correspond with expected heart rate values. The mobile computing device 720 can include a notification module 730 configured to generate a notification for display on the mobile device.

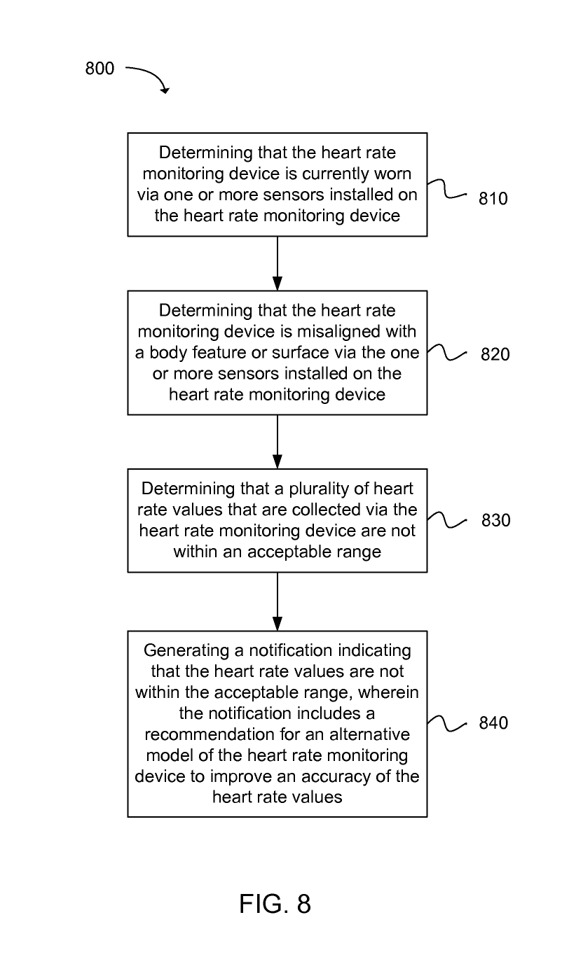

Another example provides a method 800 for generating notifications for a wearable sensor device, as shown in the flow chart in FIG. 8. The method can be executed as instructions on a machine, where the instructions are included on at least one computer readable medium or one non-transitory machine readable storage medium. The method can include the operation of determining that the wearable sensor device is currently worn via one or more sensors installed on the wearable sensor device, as in block 810. The method can include the operation of determining that the wearable sensor device is misaligned with a body feature or surface via the one or more sensors installed on the wearable sensor device, as in block 820. The method can include the operation of determining that a plurality of heart rate values that are collected via the wearable sensor device are not within an acceptable range. The method can include the operation of generating a notification indicating that the heart rate values are not within the acceptable range, wherein the notification includes a recommendation for an alternative model of the wearable sensor device to improve an accuracy of the heart rate values.

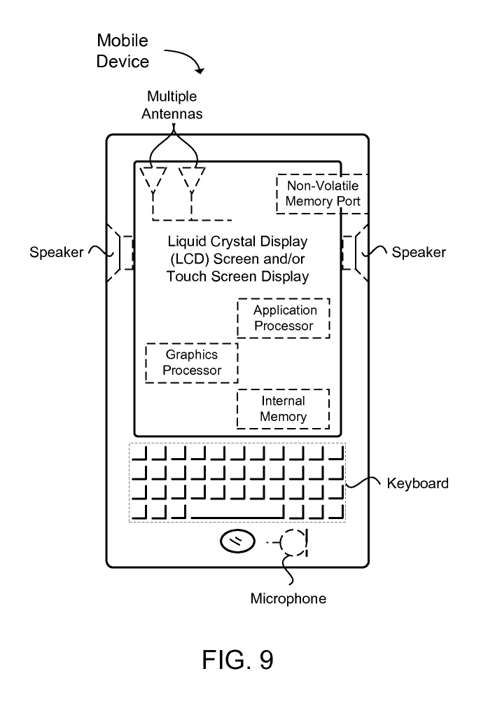

FIG. 9 provides an example illustration of a mobile computing device, such as a mobile wireless device, a mobile communication device, a tablet, a handset, or other type of wired or wireless device. The mobile computing device can include one or more antennas configured to communicate with a node, macro node, low power node (LPN), or transmission station, such as a base station (BS), an evolved Node B (eNB), a baseband unit (BBU), a remote radio head (RRH), a remote radio equipment (RRE), a relay station (RS), a radio equipment (RE), or other type of wireless wide area network (WWAN) access point. The wireless mobile computing device can be configured to communicate using at least one wireless communication standard including 3GPP LTE, WiMAX, High Speed Packet Access (HSPA), Bluetooth, and WiFi. The wireless computing device can communicate using separate antennas for each wireless communication standard or shared antennas for multiple wireless communication standards. The wireless computing device can communicate in a wireless local area network (WLAN), a wireless personal area network (WPAN), and/or a WWAN.

FIG. 9 also provides an illustration of a microphone and one or more speakers that can be used for audio input and output from the mobile computing device. The display screen can be a liquid crystal display (LCD) screen, or other type of display screen such as an organic light emitting diode (OLED) display. The display screen can be configured as a touch screen. The touch screen can use capacitive, resistive, or another type of touch screen technology. An application processor and a graphics processor can be coupled to internal memory to provide processing and display capabilities. A non-volatile memory port can also be used to provide data input/output options to a user. The non-volatile memory port can also be used to expand the memory capabilities of the mobile computing device. A keyboard can be integrated with the mobile computing device or wirelessly connected to the device to provide additional user input. A virtual keyboard can also be provided using the touch screen.

Various techniques, or certain aspects or portions thereof, can take the form of program code (i.e., instructions) embodied in tangible media, such as floppy diskettes, CD-ROMs, hard drives, non-transitory computer readable storage medium, or any other machine-readable storage medium wherein, when the program code is loaded into and executed by a machine, such as a computer, the machine becomes an apparatus for practicing the various techniques or methods recited herein. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a RAM, EPROM, flash drive, optical drive, magnetic hard drive, solid state drive, or other medium for storing electronic data. The node and wireless device can also include a transceiver module, a counter module, a processing module, and/or a clock module or timer module. One or more programs that can implement or utilize the various techniques described herein can use an application programming interface (API), reusable controls, and the like. Such programs can be implemented in a high level procedural or object oriented programming language to communicate with a computer system. However, the program(s) can be implemented in assembly or machine language, if desired. In any case, the language can be a compiled or interpreted language, and combined with hardware implementations.

It should be understood that many of the functional units described in this specification have been labeled as modules, in order to more particularly emphasize their implementation independence. For example, a module can be implemented as a hardware circuit comprising custom VLSI circuits or gate arrays, off-the-shelf semiconductors such as logic chips, transistors, or other discrete components. A module can also be implemented in programmable hardware devices such as field programmable gate arrays, programmable array logic, programmable logic devices or the like.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together, but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules, and can be embodied in any suitable form and organized within any suitable type of data structure. The operational data can be collected as a single data set, or can be distributed over different locations including over different storage devices, and can exist, at least partially, merely as electronic signals on a system or network. The modules can be passive or active, including agents operable to perform desired functions.

While the forgoing examples are illustrative of various technology principles in one or more particular applications, it will be apparent to those of ordinary skill in the art that numerous modifications in form, usage and details of implementation can be made without the exercise of inventive faculty, and without departing from the principles and concepts elaborated herein.

In an exemplary embodiment there is provided a wearable heart rate monitor comprising a heart rate detector, a position detector, a housing configured to engage a body feature or surface in a manner that allows for heart rate detection and a communication module configured to transmit heart rate and position data to a receiver.

In one example, the position detector includes an accelerometer.

In one example, the position detector includes a gyroscope.

In one example, the position detector includes a temperature sensor.

In one example, the position detector includes a proximity detector.

In one example, the communication module is further configured to transmit the heart rate and position data to the receiver to enable a computing device associated with the receiver to determine whether the wearable heart rate monitor is currently worn and aligned or misaligned with the body feature or surface based on the heart rate and position data.

In one example, the computing device is a processor located within the housing of the monitor.

In one example, the wearable heart rate monitor further comprises a signal output for signaling the user as to the alignment or misalignment of the wearable device.

In one example, the signal is an audible signal.

In one example, signal is a visual signal.

In one example, the signal is a tactile signal.

In one example, the receiver is located outside of the housing.

In one example, the housing of the wearable heart rate monitor has a shape configured to allow the wearable heart rate monitor to be inserted and held within a user's ear in order to detect heart rate information.

In one example, the wearable heart rate monitor further comprises a soft and flexible tip member configured to provide a friction fit within the user's ear.

In one example, the wearable heart rate monitor further comprises a speaker configured to broadcast an audio signal to the user's ear.

In one example, the wearable heart rate monitor comprises an input coupled to the speaker and configured to provide an electronic signal to the speaker.

In one example, the housing of the wearable heart rate monitor has a shape configured to allow the wearable heart rate monitor to be attached to a user's wrist in order to detect heart rate information.

In one example, the housing of the wearable heart rate monitor has a shape configured to allow the wearable heart rate monitor to be attached to a users' chest in order to detect heart rate information.

In one example, the wearable heart rate monitor further comprises a power source.

In one example, the wearable heart rate monitor further comprises a cord for electrically coupling the monitor to a computing device.

In one exemplary invention embodiment there is provided, a mobile device operable to detect whether a wearable sensor, such as a heart rate monitoring device is misaligned. The mobile device can receive acceleration data from the wearable heart rate monitoring device, the acceleration data including a first acceleration vector at a first time instance for the wearable heart rate monitoring device and a second acceleration vector at a second time instance for the wearable heart rate monitoring device; calculate a change in magnitude between the first acceleration vector and the second acceleration vector; calculate a misalignment vector as a difference between the first acceleration vector and the second acceleration vector; provide the change in magnitude and the misalignment vector to a classifier, wherein the classifier compares the change in magnitude and the misalignment vector to historical data to determine whether the wearable device is currently worn; and determine that the wearable heart rate monitoring device is misaligned with a body feature or surface when heart rate information collected by the wearable heart rate monitoring device does not correspond with expected heart rate values.

In one example, the classifier provides a first output value indicating that the wearable heart rate monitoring device is misaligned with the body feature or surface or a second output value indicating that the wearable heart rate monitoring device is not misaligned with the body feature or surface.

In one example, the mobile device can generate a notification when the wearable heart rate monitoring device is misaligned with the body feature or surface; and display the notification for a user associated with the mobile device.

In one example, the mobile device can provide the acceleration data through a low pass filter to extract a gravity component from the acceleration data; and provide the gravity component of the acceleration data to the classifier, wherein the classifier compares the gravity component of the acceleration data to historical data to determine whether the wearable heart rate monitoring device is currently worn.

In one example, the acceleration data used to calculate the change in magnitude and the misalignment vector is previously passed through a high pass filter in order to remove a gravity component from the acceleration data.

In one example, the mobile device can compare an average of the change in magnitude or the misalignment vector to a defined threshold; and determine that the wearable heart rate monitoring device is currently worn based on the average in relation to the defined threshold.

In one example, the acceleration data is collected via an accelerometer installed on the wearable heart rate monitoring device.

In one example, the mobile device can receive the acceleration data from the wearable heart rate monitoring device via a wired or a wireless connection.

In one example, the mobile device can detect whether the wearable heart rate monitoring device is misaligned with the body feature or surface via an application running on a mobile device.

In one example, the wearable heart rate monitoring device that sends the acceleration data to the mobile device is an in-ear heart rate monitoring headphone.

In one example, the mobile device can receive temperature data from the wearable heart rate monitoring device, wherein the wearable heart rate monitoring device collects the temperature data via a temperature sensor installed on the wearable device and provides the temperature data to the classifier, wherein the classifier compares the temperature data to historical data to determine whether the wearable heart rate monitoring device is currently worn.

In one example, the mobile device can calculate the change in magnitude (d) using |\sqrt{R₂ₓ²+R_{2y}²+R_{2z}²}−\sqrt{R₁ₓ²+R_{1y}²+R_{1z}²}|, wherein: the first acceleration vector is represented as R₁; the second acceleration vector is represented as R₂; R₁ₓ, R_{1y}, and R_{1z} are measured values associated with R₁; and R₂ₓ, R_{2y}, and R_{2z} are measured values associated with R₂.

In one example, the mobile device can calculate the misalignment vector (r) using (R₁ₓ−R₂ₓ, R_{1y}−R_{2y}, R_{1z}−R_{2z}), wherein: R₁ₓ, R_{1y}, and R_{1z} are measured values associated with the first acceleration vector (R₁); and R₂ₓ, R_{2y}, and R_{2z} are measured values associated with the second acceleration vector (R₂).

In one exemplary invention embodiment, a system is provided that comprises: a wearable heart rate monitoring device that includes a heart rate sensor and an accelerometer; and a mobile device in communication with the heart rate monitoring device. The mobile device comprises: a processor; a memory device including a data store to store a plurality of data and instructions that, when executed by the processor, cause the processor to execute: a data identification module configured to receive acceleration data for the heart rate monitoring device; a feature extraction module configured to determine one or more features of the acceleration data; a classification module configured to compare the one or more features of the acceleration data to historical data to determine whether the heart rate monitoring device is currently worn; and a misalignment module configured to determine that the wearable heart rate monitoring device is misaligned with a body feature or surface when heart rate information collected by the heart rate detector does not correspond with expected heart rate values.

In one example, the classification module is further configured to provide a first output value indicating that the wearable heart rate monitoring device is misaligned with the body feature or surface or a second output value indicating that the wearable heart rate monitoring device is not misaligned with the body feature or surface worn.

In one example, the feature extraction module is further configured to calculate a change in magnitude between a first acceleration vector and a second acceleration vector included in the acceleration data; and the classification module is further configured to compare the change in magnitude to the historical data to determine whether the heart rate monitoring device is currently worn.

In one example, the feature extraction module is further configured to calculate a change in magnitude (d) between a first acceleration vector and a second acceleration vector included in the acceleration data using |\sqrt{R₂ₓ²+R_{2y}²+R_{2z}²}−\sqrt{R₁ₓ²+R_{1y}²+R_{1z}²}|, wherein: the first acceleration vector is represented as R1; the second acceleration vector is represented as R₂; R₁ₓ, R_{1y}, and R_{1z} are measured values associated with R₁; and R₂ₓ, R_{2y}, and R_{2z} are measured values associated with R₂.

In one example, the feature extraction module is further configured to calculate a misalignment vector as a difference between a first acceleration vector and a second acceleration vector included in the acceleration data; and the classification module is further configured to compare the misalignment vector to the historical data to determine whether the heart rate monitoring device is misaligned with the body feature or surface worn.

In one example, the feature extraction module is further configured to calculate a misalignment vector (r) as a difference between a first acceleration vector and a second acceleration vector included in the acceleration data using (R₁ₓ−R₂ₓ, R_{1y}−R_{2y}, R_{1z}−R_{2z}), wherein: R₁ₓ, R_{1y}, and R_{1z} are measured values associated with the first acceleration vector (R₁); and R₂ₓ, R_{2y}, and R_{2z} are measured values associated with the second acceleration vector (R₂).

In one example, the feature extraction module is further configured to determine a gravity component from the acceleration data; and the classification module is further configured to compare the gravity component from the acceleration data to the historical data to determine whether the heart rate monitoring device is currently worn.

In one example, the mobile device further comprises a notification module configured to generate a notification for display on the mobile device, wherein notification indicates that the wearable heart rate monitoring device is misaligned with the body feature or surface worn, wherein the notification includes recommendations for wearable heart rate monitoring devices of other sizes.

In one example, the wearable heart rate monitoring device is insertable in a user's ear.

In one example, the wearable heart rate monitoring device includes a temperature sensor; the data identification module of the mobile device is further configured to receive temperature data from the temperature sensor; and the classification module of the mobile device is further configured to compare the temperature data to historical data to determine whether the wearable device is currently worn.

In one exemplary invention embodiment, a method for generating notifications for a heart rate monitoring device is provided. The method can comprise: under control of one or more computer systems configured with executable instructions: determining that the heart rate monitoring device is currently worn via one or more sensors installed on the heart rate monitoring device, using one or more processors of the computer systems; determining that the heart rate monitoring device is misaligned with a body feature or surface via the one or more sensors installed on the heart rate monitoring device, using the one or more processors of the computer systems; determining that a plurality of heart rate values that are collected via the heart rate monitoring device are not within an acceptable range, using the one or more processors of the computer systems; and generating a notification indicating that the heart rate values are not within the acceptable range, wherein the notification includes a recommendation for an alternative model of the heart rate monitoring device to improve an accuracy of the heart rate values, using the one or more processors of the computer systems.

In one example, the method further comprises providing the notification for display on a mobile device that is in communication with the heart rate monitoring device.

In one example, the heart rate monitoring device is an in-ear heart rate monitoring headphone.

In one example, the step of determining that the heart rate monitoring device is currently worn further comprises: collecting sensor data that characterizes the heart rate monitoring device from at least one of an accelerometer, a gyroscope, a temperature sensor, or a proximity sensor installed on the heart rate monitoring device; and providing the sensor data to a classifier, wherein the classifier compares the sensor data to historical data in order to determine whether the heart rate monitoring device is currently worn.

In one example, the step of determining that the heart rate monitoring device is misaligned with the body feature or surface further comprises: collecting sensor data that characterizes the heart rate monitoring device from at least one of an accelerometer, a gyroscope, a temperature sensor, a heart rate detector or a proximity sensor installed on the heart rate monitoring device; and providing the sensor data to a classifier, wherein the classifier compares the sensor data to historical data in order to determine whether the heart rate monitoring device is misaligned with the body feature or surface.

In one example, the method further comprises using sensor data collected from the one or more sensors to ensure that the heart rate monitoring device is accurately positioned with respect to the body feature or surface in a manner to enable accurate heart rate detection, wherein the sensor data includes at least one of accelerometer data, heart rate data, gyroscope data or proximity sensor data.

In one example, the method further comprises establishing a threshold level for determining whether the heart rate monitoring device is currently worn and misaligned with the body feature or surface; and comparing sensor data collected from the one or more sensors installed on the heart rate monitoring device to the threshold level.

While the forgoing examples are illustrative of the specific embodiments in one or more particular applications, it will be apparent to those of ordinary skill in the art that numerous modifications in form, usage and details of implementation can be made without departing from the principles and concepts articulated herein.

What is claimed is:

1. A wearable heart rate monitor comprising:
a heart rate detector;
a position detector;
a housing configured to engage a body feature or surface in a manner that allows for heart rate detection; and
a communication module configured to transmit heart rate and position data to a receiver.

2. The wearable heart rate monitor of claim 1, wherein the position detector includes a member selected from the group consisting of: an accelerometer, a gyroscope, a temperature sensor, a proximity detector or a combination thereof.

3. The wearable heart rate monitor of claim 1, wherein the communication module is further configured to transmit the heart rate and position data to the receiver to enable a computing device associated with the receiver to determine whether the wearable heart rate monitor is currently worn and aligned or misaligned with the body feature or surface based on the heart rate and position data.

4. The wearable heart rate monitor of claim 1, further comprising a signal output for signaling the user as to the alignment or misalignment of the wearable device.

5. The wearable heart rate monitor of claim 8, wherein the signal is an audible signal, a visual signal, or a tactile signal.

6. The wearable heart rate monitor of claim 1, wherein the housing of the wearable heart rate monitor has a shape configured to allow the wearable heart rate monitor to be inserted and held within a user's ear in order to detect heart rate information.

7. The wearable heart rate monitor of claim 6, further comprising a soft and flexible tip member configured to provide a friction fit within the user's ear.

8. The wearable heart rate monitor of claim 6, further comprising a speaker configured to broadcast an audio signal to the user's ear.

9. The wearable heart rate monitor of claim 1, wherein the housing of the wearable heart rate monitor has a shape configured to allow the wearable heart rate monitor to be attached to a user's wrist in order to detect heart rate information.

10. The wearable heart rate monitor of claim 1, wherein the housing of the wearable heart rate monitor has a shape configured to allow the wearable heart rate monitor to be attached to a users' chest in order to detect heart rate information.

11. A heart rate sensing system, comprising:
a wearable heart rate monitoring device having a heart rate detector and a position detector; and
a mobile device in communication with the heart rate monitoring device, the mobile device comprising:
a processor;
a memory device including a data store to store a plurality of data and instructions that, when executed by the processor, cause the processor to execute:
a data identification module configured to receive acceleration data for the heart rate monitoring device;
a feature extraction module configured to determine one or more features of the acceleration data; and
a classification module configured to compare the one or more features of the acceleration data to historical data to determine whether the heart rate monitoring device is currently worn; and
a misalignment module configured to determine that the wearable heart rate monitoring device is misaligned with a body feature or surface when heart rate information collected by the heart rate detector does not correspond with expected heart rate values.

12. The system of claim 11, wherein the classification module is further configured to provide a first output value indicating that the wearable heart rate monitoring device is misaligned with the body feature or surface or a second output value indicating that the wearable heart rate monitoring device is not misaligned with the body feature or surface worn.

13. The system of claim 11, wherein:
the feature extraction module is further configured to calculate a change in magnitude between a first acceleration vector and a second acceleration vector included in the acceleration data; and
the classification module is further configured to compare the change in magnitude to the historical data to determine whether the heart rate monitoring device is currently worn.

14. The system of claim 11, wherein the feature extraction module is further configured to calculate a change in magnitude (d) between a first acceleration vector and a second acceleration vector included in the acceleration data using |\sqrt{R₂ₓ²+R_{2y}²+R_{2z}²}−\sqrt{R₁ₓ²+R_{1y}²+R_{1z}²}|, wherein:
the first acceleration vector is represented as R₁;
the second acceleration vector is represented as R₂;
R₁ₓ, R_{1y}, and R_{1z} are measured values associated with R₁; and
R₂ₓ, R_{2y}, and R_{2z} are measured values associated with R₂.

15. The system of claim 11, wherein:
the feature extraction module is further configured to calculate a misalignment vector as a difference between a first acceleration vector and a second acceleration vector included in the acceleration data; and
the classification module is further configured to compare the misalignment vector to the historical data to determine whether the heart rate monitoring device is misaligned with the body feature or surface worn.

16. The system of claim 11, wherein the feature extraction module is further configured to calculate a misalignment vector (r) as a difference between a first acceleration vector and a second acceleration vector included in the acceleration data using (R₁ₓ−R₂ₓ, R_{1y}−R_{2y}, R_{1z}−R_{2z}), wherein:
R₁ₓ, R_{1y}, and R_{1z} are measured values associated with the first acceleration vector (R₁); and
R₂ₓ, R_{2y}, and R_{2z} are measured values associated with the second acceleration vector (R₂).

17. The system of claim 11, wherein:
the feature extraction module is further configured to determine a gravity component from the acceleration data; and
the classification module is further configured to compare the gravity component from the acceleration data to the historical data to determine whether the heart rate monitoring device is currently worn.

18. The system of claim 11, wherein the mobile device further comprises a notification module configured to generate a notification for display on the mobile device, wherein notification indicates that the wearable heart rate monitoring device is misaligned with the body feature or surface worn, wherein the notification includes recommendations for wearable heart rate monitoring devices of other sizes.

19. The system of claim 11, wherein the wearable heart rate monitoring device is insertable in a user's ear.

20. The system of claim 11, wherein:
the wearable heart rate monitoring device includes a temperature sensor;
the data identification module of the mobile device is further configured to receive temperature data from the temperature sensor; and
the classification module of the mobile device is further configured to compare the temperature data to historical data to determine whether the wearable device is currently worn.

21. A method for generating notifications for a heart rate monitoring device, the method comprising:
under control of one or more computer systems configured with executable instructions:
determining that the heart rate monitoring device is currently worn via one or more sensors installed on the heart rate monitoring device, using one or more processors of the computer systems;
determining that the heart rate monitoring device is misaligned with a body feature or surface via the one or more sensors installed on the heart rate monitoring device, using the one or more processors of the computer systems;
determining that a plurality of heart rate values that are collected via the heart rate monitoring device are not within an acceptable range, using the one or more processors of the computer systems; and
generating a notification indicating that the heart rate values are not within the acceptable range, wherein the notification includes a recommendation for an alternative model of the heart rate monitoring device to improve an accuracy of the heart rate values, using the one or more processors of the computer systems.

22. The method of claim 21, further comprising providing the notification for display on a mobile device that is in communication with the heart rate monitoring device.

23. The method of claim 21, wherein the heart rate monitoring device is an in-ear heart rate monitoring headphone.

24. The method of claim 21, wherein determining that the heart rate monitoring device is currently worn further comprises:
collecting sensor data that characterizes the heart rate monitoring device from at least one of an accelerometer, a gyroscope, a temperature sensor, or a proximity sensor installed on the heart rate monitoring device; and
providing the sensor data to a classifier, wherein the classifier compares the sensor data to historical data in order to determine whether the heart rate monitoring device is currently worn.

25. The method of claim 21, wherein determining that the heart rate monitoring device is misaligned with the body feature or surface further comprises:
collecting sensor data that characterizes the heart rate monitoring device from at least one of an accelerometer, a gyroscope, a temperature sensor, a heart rate detector, or a proximity sensor installed on the heart rate monitoring device; and
providing the sensor data to a classifier, wherein the classifier compares the sensor data to historical data in order to determine whether the heart rate monitoring device is misaligned with the body feature or surface.
